(19) United States
(12) Patent Application Publication
(10) Pub. No.: US 2011/0072538 A1
(43) Pub. Date: Mar. 24, 2011

(54) Use of a Chimeric Gene 4-CL Encoding 4-Coumarate:CoA Ligase in Brassicaceae

(75) Inventors: Hanzhong Wang, Hubei (CN); Xiangdong Yang, Jilin (CN); Guihua Liu, Hubei (CN); Xinfa Wang, Hubei (CN); Wei Hua, Hua (CN); Jing Liu, Hubei (CN); Qing Yang, Hubei (CN); Yuanben Zheng, Hubei (CN)

(73) Assignee: Oil Crops Research Institute Chinese Academy of Agricultural Sciences, Wuben, Hubei (CN)

(21) Appl. No.: 12/993,802

(22) PCT Filed: Nov. 25, 2008
(86) PCT No.: PCT/CN2008/073181

§ 371 Date: Nov. 19, 2010

(30) Foreign Application Priority Data
May 20, 2008 (CN) 200810047778.2

(51) Int. Cl. A01H 1/00; C12N 5/10; C07H 21/04; A01H 5/00

(52) U.S. Cl. 800/279; 435/419; 536/23.1; 536/23.2; 800/278; 800/306

(57) ABSTRACT

The use of a lignin monomer synthetic gene 4-CL encoding 4-coumarate: CoA ligase from Populus tomentosa in increasing Sclerotinia resistance and lodging resistance. The 4-CL gene is responsible for catalyzing various cinnamic acids and their derivatives to produce respective coenzyme A esters, involved in the synthesis of various downstream secondary products, and is the key gene in the regulation of lignin synthesis. Results of the study show that, increase of lignin content improves plant's resistance against pathogenic infection, enhances the stem strength of rape, and thus significantly improves the lodging resistance. Specifically, the result shows that lignin content of transgenic rape increases more than 10% compared to lignin content of recipient control; lesion extension area of transgenic plant leaves decreases around 30% compared to recipient control; and stem strength of transgenic plant increases up to around 20% compared to control.

### FIELD OF THE INVENTION

The present invention relates to the field of molecular biology. In particular, it relates to the use of this a chimeric gene encoding 4-coumarate CoA ligase (“4-CL”) in Brassicaceae plants, such as Brassica napus.

### BACKGROUND OF THE INVENTION

Lignin, as a complex phenylpropane polymer in plant, is one of the main components of mechanical tissue and cell wall of plant, and it plays an important role in aspects of plant growth and development and resistance to adversity. In the process of cell wall lignification, lignin permeates into cell wall, and fills up the cell wall skeleton, thus enhances the hardness of cell wall, and strengthens the mechanical support and compressive strength of the cell. In the interaction of host with pathogen, cell wall lignification is one of the characteristics of anti-disease response. Lignin, as an important physical anti-pathogen material in plant, can deposit on hydroxyproline-rich glycoprotein (HRGP) in cell wall, to strengthen cell wall and protect cell from pathogen infection as a structural barrier.

For the past few years, study on the regulation of lignin biosynthesis with mutants or transgenic plants mainly takes place at three levels: the first is the regulation of phenylalanine pathway, which involves 3 enzymes: PAL (phenylalanine ammonia-lyase), C4H (cinnamate 4-hydroxylase) and 4-CL (4-coumarate: CoA ligase), their expression determines the total amount of lignin; the second is the regulation of specific synthesis pathway of lignin, which mainly focuses on 3 enzymes, i.e. COMT (caffeic acid O-methyltransferase), CCoAOMT (caffeoyl-CoA O-methyltransferase) and F5H (ferulate 5-hydroxylase), expression of these enzymes has impact on lignin content and especially on specific synthesis of lignin monomer, and determines the ratio of each monomer in total lignin; the third is the regulation of enzymes downstream of the specific synthesis pathway of lignin, which includes two reductases: CAD (cinnamyl alcohol dehydrogenase) and CCR (cinnamoyl-CoA reductase), they are responsible for reducing various hydroxycinnamoyl-coenzyme A (CoA) esters to respective lignin monomers.

Phenylalanine pathway is one of the three secondary metabolism pathways of plant. A lot of secondary metabolism products of plant come from this pathway including lignin, flavanoid, anthocyanidin, phytoalexin, phenols, and signaling molecules. This pathway consists of three enzymes, PAL, C4H and 4-CL, wherein 4-CL is responsible for catalyzing cinnamic acids and their derivatives to produce respective coenzyme A esters, the thus activated substrates then enter various metabolic pathways, and take part in synthesis of various downstream secondary products.

Since 4-CL is at the metabolic branching point in the phenylalanine pathway, and is the key enzyme in the lignin monomer synthesis pathway, it is considered the ideal target gene for regulating lignin synthesis. At present, studies on 4-CL regulation are mostly aimed at reducing the activity level of endogenous 4-CL of plant by antisense RNA technique, so as to reduce lignin content, and improve paper-making quality of the wood, and feeding value of the pasture.

Lu et al. (2004, Biotechnology Letters 26, 14 pp 1147-1152) described the introduction of a gene encoding the 4-CL from Populus tomentosa under control of its own promoter in tobacco. The transgenic tobacco lines had an increased lignin content in the stem (25%) but there was no increase of lignin content in the leaves.

Research on aspects of increasing lignin content in Brassicaceae plants, and improving resistance to adverse environment by 4-CL overexpression has never been reported.

There are many genes or cDNAs encoding 4-CL protein isolated from various plants publicly available. The nucleotide sequence of the cDNA of Populus tomentosa and the amino acid sequence of the protein have been made publicly available under accession number DQ076679 and AAY84731.

There remains a need in the art to increase the resistance of Brassicaceae plants, particularly Brassica plants against fungal diseases, as well as to increase the resistance of such plants to lodging.

### SUMMARY OF THE INVENTION

An object of this invention is to provide a chimeric gene comprising a coding region of the lignin monomer synthetic gene 4-CL from Populus tomentosa under control of a heterologous plant expressible promoter, preferably a wound-inducible and/or xylem-selective promoter such as the promoter of the cinnamate-4-hydroxylase gene. The 4-CL gene is the key gene for regulating the total amount of lignin synthesized, responsible for catalyzing various cinnamic acids and their derivatives to produce respective cinnamic coenzyme A esters, and involved in synthesis of various downstream secondary products, thus increases the total amount of lignin, and improves the ability of plant to resist pathogenic infection.

Another object of this invention is to provide the use of a chimeric gene encoding 4-CL such as the 4-CL gene from Populus tomentosa, in resistance of Brassicaceae plants, such as Brassica napus, against fungal disease such as diseases caused by Sclerotinia sclerotiorum.

Yet another object of this invention is to provide the use of lignin monomer synthetic gene 4-CL from Populus tomentosa, in increasing lodging resistance of Brassica napus.

The invention also provides plants and plant cells, preferably Brassica plant cells and plants, comprising a chimeric gene comprising a DNA fragment encoding 4-CL under control of a heterologous plant-expressible promoter and methods to produce such Brassica plants.

### BRIEF DESCRIPTION OF THE DRAWINGS

FIG. 1 Picture of gel-electrophoresis of 4-CL cDNA target fragments amplified by RT-PCR.

FIG. 2 Schematic diagram of construction of the plant expression vector. Kan: kanamycin resistance, C4H: promoter fragment, 4-CL: cDNA clone of the Populus tomentosa 4-coumarate:CoA ligase is inserted downstream of the C4H promoter.

FIG. 3: Panel A: Rooted shoots of a transgenic B. napus comprising the C4H-4CL chimeric gene; Panel B: Transgenic B. napus plants transferred to soil containers.

FIG. 4 PCR results of 4-CL transgenic Brassica napus with C4H:4CL chimeric gene. Lanes 5-27 are samples from transformed plants, − is wild plant control, + is positive control. The result shows that the chimeric 4-CL gene has been successfully introduced in the majority of the transformed plant lines.

FIG. 5 Comparison of transgenic B. napus plants and control plants after inoculation with Sclerotinia sclerotiorum.

FIG. 6 Schematic representation of the stem strength of transgenic B. napus plant lines and control plants.

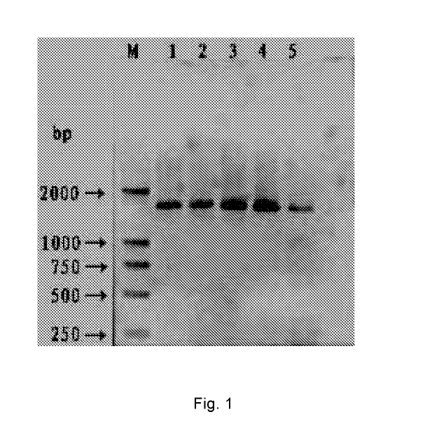

### DETAILED DESCRIPTION OF VARIOUS EMBODIMENTS OF THE INVENTION

The invention is based on the observation by the inventors that transgenic Brassica plants comprising a chimeric gene comprising a DNA fragment encoding 4-CL under control of a heterologous plant-expressible promoter (such as a plant expressible promoter driving xylem-specific transcription and/or a wound-inducible plant-expressible promoter) had an increased content of lignin, and furthermore exhibited increased resistance to infection by the fungal pathogen Sclerotinia sclerotiorum as well as increased resistance to lodging.

In a first embodiment, the invention thus provides a method to increase resistance against fungal disease or to increase lodging resistance in a Brassica plant comprising the step of providing cells of said Brassica plant with a chimeric gene, said chimeric gene comprising a nucleotide sequence encoding a 4-coumarate-CoA ligase, preferably a 4-coumarate-CoA ligase from plant origin, operably linked to a heterologous plant-expressible promoter and a transcription termination and polyadenylation region.

As used herein “4-coumarate-CoA ligase” refers to an enzyme (EC 6.2.1.12) that catalyzes the chemical reaction:

ATP+4-coumarate+CoA [symbol] AMP+diphosphate+4-coumaroyl-CoA

This enzyme belongs to the family of ligases, specifically those forming carbon-sulfur bonds as acid-thiol ligases. The systematic name of this enzyme class is 4-coumarate:CoA ligase (AMP-forming). Other names in common use include 4-coumaroyl-CoA synthetase, p-coumaroyl CoA ligase, p-coumaryl coenzyme A synthetase, p-coumaryl-CoA synthetase, p-coumaryl-CoA ligase, feruloyl CoA ligase, hydroxycinnamoyl CoA synthetase, 4-coumarate:coenzyme A ligase, caffeolyl coenzyme A synthetase, p-hydroxycinnamoyl coenzyme A synthetase, feruloyl coenzyme A synthetase, sinapoyl coenzyme A synthetase, 4-coumaryl-CoA synthetase, hydroxycinnamate:CoA ligase, p-coumaryl-CoA ligase, p-hydroxycinnamic acid:CoA ligase, and 4CL. This enzyme participates in phenylpropanoid biosynthesis.

In one embodiment, a DNA fragment encoding 4CL from Populus tomentosa is used, such as a DNA fragment comprising the nucleotide sequence of SEQ ID NO. 1 from nucleotide 11 to nucleotide 1621. However, having read the current invention, the person skilled in the art will realize that other DNA fragments encoding other 4CL enzymes (from plant origin) can be used to similar effect.

Alternative 4-CL encoding nucleic acids suitable for the methods and means of the current invention are nucleotide sequences encoding 4-CL proteins having an amino acid sequence which may be found in amino acid sequence databases such as the entries with the following identification numbers: AAB42382 (Pinus taeda); AAB42383 (Pinus taeda); AAC24503 (Populus tremuloides); AAC24504 (Populus tremuloides); AAF37732 (Lolium perenne); AAF37733 (Lolium perenne); AAF37734 (Lolium perenne); AAG46175 (Oryza sativa); AAG50881 (Arabidopsis thaliana); AAL24191 (Arabidopsis thaliana); AAL30738 (Oryza sativa); AAL35216 (Amorpha fruticosa); AAL90967 (Arabidopsis thaliana); AAL91633 (Arabidopsis thaliana); AAM19949 (Arabidopsis thaliana); AAM91412 (Arabidopsis thaliana); AAN18181 (Arabidopsis thaliana); AA064847 (Arabidopsis thaliana); AAQ56837 (Arabidopsis thaliana); AAS82605 (Zea mays); AAT02218 (Agastache rugosa); AAW66826 (Zingiber officinale); ABB90402 (Leucaena leucocephala); ABI26719 (Hibiscus cannabinus); ABJ98946 (Arabidopsis thaliana); ABL01799 (Leucaena leucocephala); ABY89349 (Pinus taeda); ABY89350 (Pinus taeda); ABY89351 (Pinus taeda); ABY89352 (Pinus taeda); ABY89353 (Pinus taeda); ABY89354 (Pinus taeda); ABY89355 (Pinus taeda); ABY89356 (Pinus taeda); ABY89357 (Pinus taeda); ABY89358 (Pinus taeda); ABY89359 (Pinus taeda); ABY89360 (Pinus taeda); ABY89361 (Pinus taeda); ABY89362 (Pinus taeda); ABY89363 (Pinus taeda); ABY89364 (Pinus taeda); ABY89365 (Pinus taeda); ABY89366 (Pinus taeda); ABY89367 (Pinus taeda); ABY89368 (Pinus taeda); ABY89369 (Pinus taeda); ABY89370 (Pinus taeda); ABY89371 (Pinus taeda); ABY89372 (Pinus taeda); ABY89373 (Pinus taeda); ABY89374 (Pinus taeda); ABY89375 (Pinus taeda); ABY89376 (Pinus taeda); ABY89377 (Pinus taeda); ABY89378 (Pinus taeda); ABY89379 (Pinus taeda); ABY89380 (Pinus taeda); ABY89381 (Pinus radiata); ABY89382 (Pinus radiata); ABY89383 (Pinus sylvestris); ABY89384 (Pinus sylvestris); ABY89385 (Pinus elliottii); ABY89386 (Pinus elliottii); CAK22404 (Picea abies); P0C5B6 (Arabidopsis thaliana); P17814 (Oryza sativa); P41636 (Pinus taeda); Q0DV32 (Oryza sativa); Q10S72 (Oryza sativa); Q336M7 (Oryza sativa); Q3E6Y4 (Arabidopsis thaliana); Q42524 (Arabidopsis thaliana); Q42982 (Oryza sativa); Q67W82 (Oryza sativa); Q69RG7 (Oryza sativa); Q6ETN3 (Oryza sativa); Q6YYZ2 (Oryza sativa); Q6ZAC1 (Oryza sativa); Q7F1X5 (Oryza sativa); Q7XXL2 (Oryza sativa); Q84P21 (Arabidopsis thaliana); Q84P23 (Arabidopsis thaliana); Q84P24 (Arabidopsis thaliana); Q84P25 (Arabidopsis thaliana); Q84P26 (Arabidopsis thaliana); Q8GVF9 (Oryza sativa); Q8RU95 (Oryza sativa); Q9LQ12 (Arabidopsis thaliana); Q9LU36 (Arabidopsis thaliana); Q9M0X9 (Arabidopsis thaliana); Q9S725 (Arabidopsis thaliana); Q9S777 (Arabidopsis thaliana); AAL56850 (Populus tomentosa); AAL02145 (Populus tomentosa); AAL02144 (Populus tomentosa); AAY84731 (Populus tomentosa); AAS88873 (Populus tomentosa) (all sequences herein incorporated by reference).

Examples of nucleotide sequences encoding 4-CL proteins, suitable for the methods of the current invention can be found in nucleotide sequence databases as the entries with the following identification numbers: EU224389 (Ruta graveolens); EU224388 (Ruta graveolens); AL161502 (Arabidopsis thaliana); NC_{—}003070 (Arabidopsis thaliana); NC_{—}003076 (Arabidopsis thaliana); NC_{—}003075 (Arabidopsis thaliana); NC_{—}003074 (Arabidopsis thaliana); NC_{—}003071 (Arabidopsis thaliana chromosome); EU196764 (Ruta graveolens); EU196763 (Ruta graveolens); BV728707 (Pinus taeda); X05353 (Petroselinum crispum); X13325 (Petroselinum crispum); DQ093070 (Populus deltoides); NM_{—}101901 (Arabidopsis thaliana); NM_{—}179513 (Arabidopsis thaliana); NM_{—}123172 (Arabidopsis thaliana); NM_{—}113019 (Arabidopsis thaliana); NM_{—}105180 (Arabidopsis thaliana); NM_{—}104972 (Arabidopsis thaliana); NM_{—}101898 (Arabidopsis thaliana); NM_{—}001084228 (Arabidopsis thaliana); NM_{—}202143 (Arabidopsis thaliana); NM_{—}116755 (Arabidopsis thaliana); NM_{—}113018 (Arabidopsis thaliana); NM_{—}101899 (Arabidopsis thaliana); NM_{—}125733 (Arabidopsis thaliana); NM_{—}179462 (Arabidopsis thaliana); NM_{—}104046 (Arabidopsis thaliana); NM_{—}101900 (Arabidopsis thaliana); NM_{—}113051 (Arabidopsis thaliana); NM_{—}118019 (Arabidopsis thaliana); BV728981 (Pinus taeda); EU603323 (Populus trichocarpa); NC_{—}008401 (Oryza sativa (japonica cultivar-group)); NM_{—}001068470 (Oryza sativa (japonica cultivar-group)); NM_{—}001067888 (Oryza sativa(japonica cultivar-group)); NC_{—}008399 (Oryza sativa (japonica cultivar-group)); NM_{—}001064787 (Oryza sativa (japonica cultivar-group)); NC_{—}008397 (Oryza sativa(japonica cultivar-group)); NM_{—}001060839 (Oryza sativa (japonica cultivar-group)); NM_{—}001058971 (Oryza sativa (japonica cultivar-group)); NC_{—}008396 (Oryza sativa(japonica cultivar-group)); NM_{—}001055527 (Oryza sativa (japonica cultivar-group)); NM_{—}001055402 (Oryza sativa (japonica cultivar-group)); NC_{—}008395 (Oryza sativa(japonica cultivar-group)); NM_{—}001054354 (Oryza sativa (japonica cultivar-group)); NM_{—}001052604 (Oryza sativa (japonica cultivar-group)); NC_{—}008394 (Oryza sativa (japonica cultivar-group)); NM_{—}001051646 (Oryza sativa (japonica cultivar-group)); AB234050 (Oryza sativa); NM_{—}001111788 (Zea mays); EF589315 (Oryza sativa (indica cultivar-group)); EF589314 (Oryza sativa (indica cultivar-group)); EF589313 (Oryza sativa (indica cultivar-group)); EF589285 (Oryza sativa (indica cultivar-group)); EF589284 (Oryza sativa (indica cultivar-group)); DQ400697 (Arnebia euchroma); AP008214 (Oryza sativa (japonica cultivar-group)); AP008212 (Oryza sativa (japonica cultivar-group)); AP008210 (Oryza sativa (japonica cultivar-group)); AP008209 (Oryza sativa (japonica cultivar-group)); AP008208 (Oryza sativa (japonica cultivar-group)); AP008207 (Oryza sativa); AY237164 (Salvia miltiorrhiza); AL161549 (Arabidopsis thaliana); AL132967 (Arabidopsis thaliana); AL021711 (Arabidopsis thaliana); X52623 (Oryza sativa); X13324 (Petroselinum crispum); X69954 (Glycine max L.); AF466202 (Zea mays); AC079736 (Oryza sativa); DQ147001 (Eucalyptus camaldulensis); DQ076679 (Populus tomentosa); X05351 (Petroselinum crispum); X05350 (Petroselinum crispum); AB166768 (Scutellaria baicalensis); AB166767 (Scutellaria baicalensis); AC079830 (Oryza sativa); AY566301 (Zea mays); AB007649 (Arabidopsis thaliana); AC073867 (Oryza sativa); AC079887 (Oryza sativa); AC078840 (Oryza sativa); AC022457 (Oryza sativa); AC025783 (Oryza sativa); AC068924 (Oryza sativa); AC084320 (Oryza sativa); AC091247 (Oryza sativa); AC091123 (Oryza sativa); AC087192 (Oryza sativa); AC024594 (Oryza sativa); AC084406 (Oryza sativa); AC025294 (Arabidopsis thaliana); AC018727 (Oryza sativa); AC026815 (Oryza sativa); AC069145 (Oryza sativa); AC051634 (Oryza sativa); AC027665 (Arabidopsis thaliana); BX784388 (Pinus pinaster); AY376735 (Arabidopsis thaliana); AY376734 (Arabidopsis thaliana); AY376733 (Arabidopsis thaliana); AY376732 (Arabidopsis thaliana); AY376731 (Arabidopsis thaliana); AY376730 (Arabidopsis thaliana); AY376729 (Arabidopsis thaliana); AY376728 (Arabidopsis thaliana); BT010394 (Arabidopsis thaliana); AY163489 (Nicotiana sylvestris); BT005912 (Arabidopsis thaliana); AY163490 (Nicotiana sylvestris); AF002257 (Glycine max); AF150686 (Solanum tuberosum); BT000614 (Arabidopsis thaliana); AY133582 (Arabidopsis thaliana); AY095992 (Arabidopsis thaliana); AF279267 (Glycine max); AY075622 (Arabidopsis thaliana); AF002259 (Glycine max); AY090306 (Arabidopsis thaliana); AF314180 (Populus tomentosa); AF435968 (Amorpha fruticosa); AY058083 (Arabidopsis thaliana); AY043495 (Populus tomentosa); AY043494 (Populus tomentosa); AF283552 (Populus balsamifera subsp. trichocarpa x Populus deltoides); AF008184 (Populus x generosa); AF144503 (Pinus armandii); AF144502 (Pinus armandii); AF144501 (Pinus armandii); D49367 (Lithospermum erythrorhizon); AF239687 (Rubus idaeus); AF239686 (Rubus idaeus); AF239685 (Rubus idaeus); AF052223 (Lolium perenne); AF052222 (Lolium perenne); AF052221 (Lolium perenne); U18675 (Arabidopsis thaliana); AF106088 (Arabidopsis thaliana); AF106087 (Arabidopsis thaliana); AF106085 (Arabidopsis thaliana); AF106084 (Arabidopsis thaliana); AF041051 (Populus tremuloides); AF041050 (Populus tremuloides); AF041049 (Populus tremuloides); D49366 (Lithospermum erythrorhizon); X05352 (Petroselinum crispum); L43362 (Oryza sativa); M62755 (Solanum tuberosum) (all sequences herein incorporated by reference).

Nucleotide sequences of partial cDNA clones encoding 4-CL proteins (which can be used to isolate full length cDNA clones, the latter suitable for use according to the current invention) can also be found in nucleotide sequence databases as the entries with the following identification numbers: AF207575 (Brassica oleracea); AF207574 (Brassica rapa); AF207573 (Brassica rapa); AF207572 (Brassica oleracea); AF207571 (Brassica rapa); AF207570 (Brassica rapa); AF207569 (Brassica napus); AF207568 (Brassica napus); AF207567 (Brassica napus); AF207566 (Brassica napus); AF207565 (Brassica napus); AF207564 (Brassica napus); EU183423 (Thujopsis dolabrata); EU183422 (Thuja occidentalis); EU183421 (Thuja sutchuenensis); EU183420 (Thuja standishii); EU183419 (Thuja koraiensis); EU183418 (Thuja plicata); EU183417 (Thuja plicata); AF150687 (Solanum tuberosum); AF270934 (Rubus idaeus); AF270933 (Rubus idaeus); AF144529 (Cedrus atlantica); AF144528 (Pseudolarix amabilis); AF144527 (Pseudolarix amabilis); AF144526 (Tsuga canadensis); AF144525 (Tsuga canadensis); AF144524 (Tsuga mertensiana); AF144523 (Nothotsuga longibracteata); AF144522 (Keteleeria evelyniana); AF144521 (Abies beshanzuensis); AF144520 (Abies beshanzuensis); AF144519 (Abies beshanzuensis); AF144518 (Abies holophylla); AF144517 (Abies holophylla); AF144516 (Abies firma); AF144515 (Abies firma); AF144514 (Abies firma); AF144513 (Larix gmelini); AF144512 (Larix gmelini); AF144511 (Pseudotsuga sinensis); AF144510 (Pseudotsuga sinensis); AF144509 (Pseudotsuga sinensis); AF144508 (Pseudotsuga menziesii); AF144507 (Pseudotsuga menziesii); AF144506 (Pseudotsuga menziesii); AF144505 (Cathaya argyrophylla); AF144504 (Picea smithiana); AF144500 (Pinus banksiana); AF144499 (Pinus banksiana); AY224135 (Pelargonium x hortorum); AY764488 (Pinus taeda); AY764487 (Pinus taeda); AY764486 (Pinus taeda); AH014229 (Pinus taeda); AY764485 (Pinus taeda); AY764484 (Pinus taeda); AY764483 (Pinus taeda); AH014228 (Pinus taeda); AY764482 (Pinus taeda); AY764481 (Pinus taeda); AY764480 (Pinus taeda); AY575768 (Populus tomentosa); D50034 (Nicotiana tabacum); D50033 (Nicotiana tabacum); EU280886 (Larix sibirica); EU280885 (Larix sukaczewii); EU280884 (Larix sukaczewii); EU280883 (Larix sukaczewii); EU280882 (Larix sukaczewii); EU280881 (Larix sukaczewii); EU280880 (Larix sukaczewii); EU280879 (Larix sukaczewii); EU280878 (Larix sukaczewii); EU280877 (Larix sukaczewii); EU280876 (Larix sukaczewii); EU280875 (Larix sukaczewii); EU280874 (Larix sukaczewii); EU280873 (Larix sibirica); EU280872 (Larix sibirica); EU280871 (Larix sibirica); EU280870 (Larix sibirica); EU280869 (Larix sibirica); EU280868 (Larix sibirica); EU280867 (Larix sibirica); EU280866 (Larix kaempferi); EU280865 (Larix kaempferi); EU280864 (Larix gmelinii); EU280863 (Larix gmelinii); EU280862 (Larix gmelinii var. Olgensis); EU280861 (Larix gmelinii var. Olgensis); EU280860 (Larix gmelinii var. Olgensis); EU280859 (Larix gmelinii var. Olgensis); EU280858 (Larix gmelinii var. Olgensis); EU280857 (Larix gmelinii var. Olgensis); EU280856 (Larix kamtschatica); EU280855 (Larix kamtschatica); EU280854 (Larix kamtschatica); EU280853 (Larix gmelinii); EU280852 (Larix gmelinii) var. japonica; EU280851 (Larix gmelinii); EU280850 (Larix gmelinii var. Japonica); EU280849 (Larix gmelinii); EU280848 (Larix gmelinii); EU280847 (Larix cajanderi); EU280846 (Larix cajanderi); EU280845 (Larix cajanderi); EU280844 (Larix cajanderi); EU280843 (Larix cajanderi); EU280842 (Larix cajanderi); EU280841 (Larix cajanderi); EU392783 (Pinus elliottii); EU392782 (Pinus elliottii); EU392781 (Pinus sylvestris); EU392780 (Pinus sylvestris); EU392779 (Pinus radiata); EU392778 (Pinus radiata); AY832332 (Pseudotsuga menziesii var. Menziesii); AY832331 (Pseudotsuga menziesii var. Menziesii); AY832330 (Pseudotsuga menziesii var. Menziesii); AY832329 (Pseudotsuga menziesii var. Menziesii); AY832328 (Pseudotsuga menziesii var. Menziesii); AY832327 (Pseudotsuga menziesii var. Menziesii); AY832326 (Pseudotsuga menziesii var. Menziesii); AY832325 (Pseudotsuga menziesii var. Menziesii); AY832324 (Pseudotsuga menziesii var. Menziesii); AY832323 (Pseudotsuga menziesii var. Menziesii); AY832322 (Pseudotsuga menziesii var. Menziesii); AY832321 (Pseudotsuga menziesii var. Menziesii); AY832320 (Pseudotsuga menziesii var. Menziesii); AY832319 (Pseudotsuga menziesii var. Menziesii); AY876936 (Zingiber officinale); U61383 (Oryza rufipogon); DQ983412 (Pinus longaeva); DQ983411 (Pinus balfouriana); DQ983410 (Pinus balfouriana); DQ983409 (Pinus balfouriana); DQ983408 (Pinus balfouriana); DQ983407 (Pinus balfouriana); DQ223433 (Liriodendron tulipifera); DQ667963 (Vitis vinifera); EF214739 (Linum usitatissimum); EF214738 (Linum usitatissimum); EF214737 (Linum usitatissimum); DQ840569 (Hibiscus cannabinus); DQ267975 (Leucaena leucocephala) (all sequences herein incorporated by reference).

In the chimeric genes according to the current invention, the 4-CL coding region is operably linked to a heterologous plant-expressible promoter.

As used herein, the term “promoter” denotes any DNA which is recognized and bound (directly and indirectly) by a DNA-dependent RNA-polymerase during initiation of transcription. A promoter includes the transcription initiation site, and binding sites for transcription initiation factors and RNA polymerase, and can comprise various other sites (e.g. enhancers), at which gene expression regulatory proteins may bind.

The term “regulatory region”, as used herein, means any DNA, that is involved in driving transcription and controlling (i.e. regulating) the timing and level of transcription of a given DNA sequence, such as a DNA coding for a protein or polypeptide. For example, a 5′ regulatory region (or “promoter region”) is a dNA sequence located upstream (i.e. 5′) of a coding sequence and which comprises the promoter and the 5′-untranslated leader sequence. A 3′ regulatory region is a DNA sequence located downstream (i.e. 3′) of the coding sequence and comprises suitable transcription termination and or regulation signals, including one or more polyadenylation signals.

As used herein, the term “plant-expressible promoter” means a DNA sequence that is capable of controlling (initiating) transcription in a plant cell. This includes any promoter of plant origin, but also any promoter of non-plant origin which is capable of directing transcription in a plant cell, i.e. certain promoters of viral or bacterial origin such as the CaMV35S promoter (Hapster et al. 1988, Mol. Gen. Genet. 212, 182-190), the subterranean clover virus promoter No 4 or No 7 (WO9606932) or T-DNA gene promoters.

A “heterologous” promoter as used herein refers to a promoter other than the promoter which is naturally associated with the coding region for the 4-CL nucleotide sequence used in accordance with this invention.

A “xylem selective promoter” as used herein is a plant expressible promoter that is preferentially, but not necessarily exclusively driving transcription in xylem tissue of the plant. As an example of such a xylem selective promoter, the promoter region of the Populus tomentosa cinnamate-4-hydroxylase (“C4H”) gene was used (see also SEQ ID NO. 3) (Zhao et al., 2005 Plant Science 168, 1157-1162). Other xylem selective promoters include the promoter described in US patent application 20070180580, the promoters from the genes encoding xylem peptidase XCP1 and XCP2 as described in Funk et al. 2002, Plant Physiology 128 (1) 84-94, the promoters described in US patent application 20080196125, the promoter of the glycin rich protein 1.8 from Sopho japonica as described by Lu et al. Plant Growth Regulation Volume 41 (3) pp 279-286.

As used herein a “wound-inducible promoter” is a promoter which drives a higher level of expression in plant tissue surrounding lesions than in intact plant tissues. The promoter region of the Populus tomentosa cinnamate-4-hydroxylase (“C4H”) has been described to be wound-inducible (Zhao et al., 2005 Plant Science 168, 1157-1162). However, wound-inducible promoters are well defined in the art and also include the promoters form potato proteinase-inhibitor genes (pin1 and pin2) and the like.

As used herein, “providing a plant cell with a chimeric gene” includes methods of introducing a chimeric plant cell transiently or stably into a plant cell using well established transformation methods. However a plant cell can also be provided with a chimeric gene through crossing and breeding steps with a transgenic plant comprising such a chimeric gene.

To realize the above objects, the following technical measures are employed to obtain lignin monomer synthetic gene 4-CL from Populus tomentosa:

1) Upon searching for the published poplar 4-CL gene sequence in NCBI database, Oligo6.0 software (commercially purchased, sic passim) is used to design primers at both sides of gene encoding sequence (forward primer: [5′-ATGAATCCACAAGAAGAATTCATC-3′] (SEQ ID NO. 4); and reverse primer: [5′-TTATATGCCTGCCAACTTTTCTTTCAG-3′] (SEQ ID NO. 5).
2) An RT-PCR amplification is performed using first-strand of cDNA as template, and the fragment resulted from the amplifications sequenced to obtain 4-CL gene sequence. The base sequence of the DNA molecule is shown in SEQ ID NO. 1.

Lignin monomer synthetic gene 4-CL from Populus tomentosa is obtained by the above method. Transgenic Brassica napus with elevated 4-CL gene expression is provided, characterized in that: the transgenic plant has its lignin content increased more than 10% compared to recipient control (non-transgenic plant); resistance thereof to Sclerotinia sclerotiorum and lodging improved; lesion extension area thereof decreased around 30% compared to recipient control (non-transgenic plant); and stem strength thereof increased around 20% compared to control (non-transgenic plant).

In the present invention, the following technical solution is used for the above-mentioned purpose. A plasmid expression vector pBI121 (commercially purchased from Institute of Genetics and Developmental Biology, Chinese Academy of Sciences (CAS)), is provided which contains specific expression promoter (C4H promoter, specifically expressed in xylem) and translation control elements.

In another aspect of the present invention, there provides a host bacteria that can be expressed in plants, preferably Agrobacterium in the present invention, and more preferably Agrobacterium tumefaciens LBA4404 (commercially purchased from Institute of Genetics and Developmental Biology, CAS).

Methods for introducing the DNA vector comprising the chimeric gene according to the invention into plant cells, particularly into Brassicaceae plant cells are well established in the art and include gene gun bombardment methods, and Agrobacteria-mediated introduction method. A recombinant DNA comprising a chimeric gene according to the invention can be stably incorporated in the nuclear genome of a cell of a plant, particularly a plant that is susceptible to Agrobacterium-mediated transformation. Gene transfer can be carried out with a vector that is a disarmed Ti-plasmid, comprising a chimeric gene of the invention, and harbored by Agrobacterium. This transformation can be carried out using the procedures described, for example, in EP 0,116,718. Ti-plasmid vector systems comprise a chimeric gene between the T-DNA border sequences, or at least to the left of the right T-DNA border. Alternatively, any other type of vector can be used to transform the plant cell, applying methods such as direct gene transfer (as described, for example, in EP 0,233,247), pollen-mediated transformation (as described, for example, in EP 0,270,356, WO85/01856 and U.S. Pat. No. 4,684,611), plant RNA virus-mediated transformation (as described, for example, in EP 0,067,553 and U.S. Pat. No. 4,407,956), liposome-mediated transformation (as described, for example, in U.S. Pat. No. 4,536,475), and the like. Agrobacterium mediated introduction is preferred in the present invention, such as Agrobacterium LBA4404 mediated introduction into Brassica napus cotyledon petiole. A method for Agrobacterium mediated transformation of Brassica plants is described in De Block et al. 1989 Plant Physiol. 914: 694-701, entitled “Transformation of Brassica napus and Brassica oleracea Using Agrobacterium tumefaciens and the Expression of the bar and neo Genes in the Transgenic Plants”

The present invention provides a method for preparing transgenic Brassica napus which can overexpress Populus tomentosa 4-CL gene, characterized in that the lignin content of Brassica napus increases, and the resistance to Sclerotinia sclerotiorum and lodging improves, which method comprises:

1) lignin monomer synthetic gene 4-CL from Populus tomentosa, obtained from cloning, is ligated to plasmid pBI121, forming a vector capable of transforming or expressing under the control of C4H promoter, which is specifically expressed in xylem (pBI121-C4H: 4CL);
2) Agrobacterium tumefaciens LBA4404 is transformed with the vector prepared in step 1) (pBI121-C4H: 4CL), then used to co-infect a Brassica napus plant;
3) a positive plant comprising the chimeric gene is selected.

In the present invention, (1) Populus tomentosa, a unique Chinese tree species is used. Based on the published conserved sequence of poplar 4-CL gene, specific primers at conserved region flanking both sides of gene encoding sequence are designed. An RT-PCR amplification is performed, and the gene sequence obtained is shown in SEQ ID NO. 1. PCR product of C4H promoter and PBI121 plasmid are respectively digested by both restriction enzymes HindIII/BamHI. The digested fragment of PCR product and the longer fragment of digested PBI121 plasmid are ligated, and the resulting reaction product is used to transform competent cells DH5α (commercially obtained), and amplified. Then 4-CL gene is introduced into vector PBI121 to replace the original GUS sequence in PBI121, while the 35S promoter sequence formerly in the vector is replaced by C4H promoter sequence. The constructed plant expression vector is renamed as pBI-C4H: 4-CL. (2) Agrobacterium tumefaciens LBA4404 is transformed with vector containing 4-CL gene, and grown in YEP liquid medium (tryptone 10 g, yeast extract 10 g, sodium chloride 5 g); then aseptic seedling is soaked in Agrobacterium suspension. (3) Positive clones are screened in kanamycin (Kan) selective medium.

In a specific example of the present invention, the aseptic seedling of the plant used was Brassica napus. Brassica napus plants with lignin monomer synthetic gene 4-CL expressed are selected in selective medium. The plants show lignin content increase of more than 10%, and their resistance to lodging and to Sclerotinia sclerotiorum increase around 20% and 30%, respectively.

The term “transgenic plant” used in this invention refers to those plants containing an inserted gene, wherein the expression of said gene can be stably enhanced or inhibited, so as to generate a specific biological trait.

The term “plant cell” used in this invention refers to all type of plant cells including but not limited to various immature embryo, callus or suspension cells (e.g., those of mature seed embryo, immature embryo, young ear, anther, coleoptile or young leaves), or protoplasts (mesophyll cell) of a plant. These plant cells can all regenerate to a plant under appropriate conditions.

Methods for cloning lignin monomer synthetic gene 4-CL from Populus tomentosa according to the present invention, are common methods used in the field. Methods for extracting mRNA include many well-founded techniques (such as using TRIzol Reagent, Invetrogen), and mRNA extraction kit can be, for example, commercially obtained. Construction of cDNA library is also a commonly-used molecular biology technique. Methods for constructing the vector described in the present invention and introducing the vector to plant are also commonly-used methods in the field. The plasmids involved (such as plasmid expression vector pBI121), and media for transformation (such as Agrobacterium tumefaciens LBA44040 and reagents used) are commercially available.

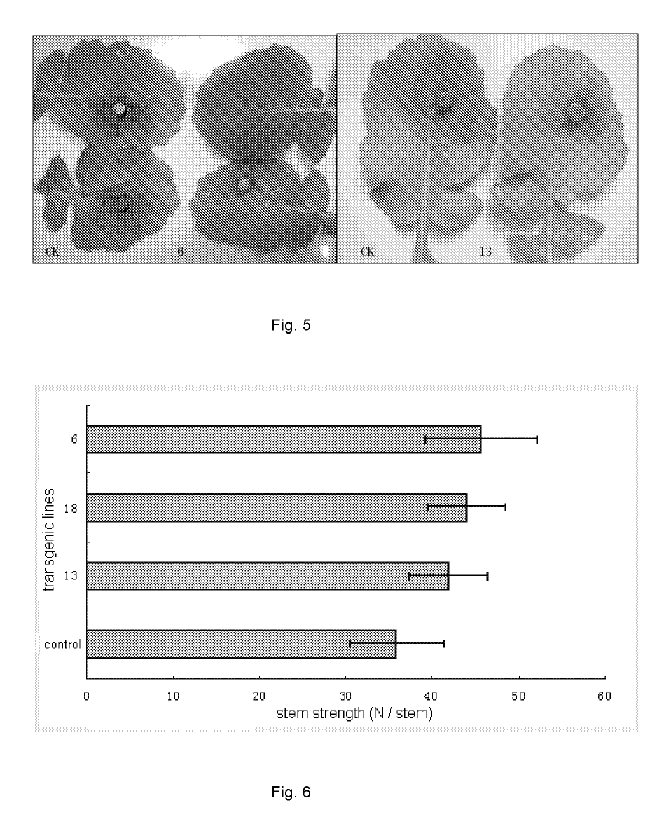

The present invention is the first to disclose the use of a DNA comprising sequence of lignin monomer synthetic gene 4-CL from Populus tomentosa. As lignin monomer synthetic gene 4-CL is at the metabolic branching point in Phenylalanine pathway, encoding the key enzyme of lignin monomer synthesis pathway, and it's considered the ideal target gene for regulating lignin synthesis. The present invention is the first to use transgenic technology to regulate synthesis of the total amount of Brassica napus lignin in order to increase the lignin content. 4-CL is responsible for catalyzing various cinnamic acids and their derivatives to produce respective coenzyme A esters, involved in synthesis of downstream secondary product, and increases the total amount of lignin. Experimental results of the present invention shows that, lignin content of transgenic Brassica napus increases compared to recipient control (non-transgenic plant), Brassica napus line with highest increase shows an increase magnitude of 13.41% (see table 1); lesion extension area of transgenic plant decreases compared to recipient control (non-transgenic plant), the highest decrease magnitude is around 30% (see FIG. 5); stem strength generally increases around 20% compared to control (non-transgenic plant) (see FIG. 6), and it significantly improves the resistance to Sclerotinia sclerotiorum and lodging in Brassica napus. This method sheds new light on study of crop breeding and resistance to disease and to lodging.

Preferred Brassicaceae to be treated in accordance with this invention, besides Brassica napus, include Brassica juncea, Brassica oleraceae, Brassica carinata, Brassica nigra, Brassica campestris and the like, and any intergenic crosses or synthetic varieties thereof. Other Brassicaceae, which may be treated according to the methods of the inventions include Brassica cretica (mustard), Brassica elongata (elongated mustard), Brassica narinosa (broadbeaked mustard), Brassica nigra (black mustard), Brassica rapa (field mustard), Brassica rupstris (mustard), Brassica toumefortii (Asian mustard). Brassica napus (2n=38, genome AACC) is an amphidiploid species, which originated from a spontaneous hybridization of Brassica rapa L. (syn. B. campestris; 2n=20, AA) and Brassica oleracea L. (2n=18, CC). B. napus contains the complete chromosome sets of these two diploid genomes.

As used herein, “a plant from the family Brassicaceae” or “a Brassicaceae plant” is a plant which according to the current botanical standard would be classified into the family Brassicaceae (formerly Cruciferaeae). Brassicaceae (Mustard) family members are easy to distinguish. They are annual or perannual plants with alternate leaves without stipules and posses simple inflorescence or branched racemes. The flowers are bilaterally symmetrical and hypogynous. With few exceptions, the flowers have 4 petals (free) alternating with 4 sepals (free); 6 stamens (4 long and 2 short), an ovary of 2 united carpels with partial placenta, 2 locular through the formation of a membranous false septum; fruit is a dehiscent capsule opening by 2 valves. Brassicaceae include inter alia the following genera: Sisymbrium, Descurania, Alliaria, Arabidopsis, Myagrum, Isatis, Bunia, Elysium, Hesperis, Malcolmia, Matthiola, Chorispora, Euclidium, Barbarea, Rorippa, Armoracia, Nasturtium, Dentaria, Cardamine, Cardaminopsis, Arabis, Lunaria, Alyssum, Berteroa, Lobularia, Draba, Erophila, Cochlearia, Camelina, Neslia, Capsella, Hornungia, Thlsapi, Iberis, Lepidium, Cardaria, Coronopus, Subularia, Conringia, Diplotaxis, Brassica, Sinapsis, Eruca, Erucastrum, Coincya, Hirschfeldia, Cakile, Rapistum, Crambe, Enarthrocarpus, Rhaphanus and Clausia.

“Brassica plant” as used herein refers to a plant from the genus Brassica and includes the species Brassica napus, Brassica oleracea, Brassica rapa, Brassica junceae and Brassica carinata.

“Oilseed rape” as used herein, should be understood to include the species Brassica napus, Brassica junceae and Brassica campestris.

It is also an object of the invention to provide plant cells and plants containing the chimeric genes or the RNA molecules according to the invention. Gametes, seeds (including crushed seeds and seed cakes), embryos, either zygotic or somatic, progeny or hybrids of plants comprising the chimeric genes of the present invention, which are produced by traditional breeding methods are also included within the scope of the present invention.

The plants obtained by the methods described herein may be further crossed by traditional breeding techniques with other plants to obtain fungal disease resistant or lodging resistant progeny plants comprising the chimeric genes of the present invention.

The following non-limiting Examples describe chimeric genes comprising a coding region encoding a 4-CL enzyme, and the use thereof in Brassica plants. Unless otherwise specified, standard conditions are used as described in Sambrook et al., Molecular Cloning: A Laboratory Manual (New York: Cold Spring Harbor Laboratory Press, 1989), in Draper, J et al. (Blackwell Scientific Publications, 1988), Volumes 1 and 2 of Ausubel et al. (1994) Current Protocols in Molecular Biology, Current Protocols, USA, or as suggested by the manufacturer of the reagents and kits involved. Standard materials and methods for plant molecular work are described in Plant Molecular Biology Labfax (1993) by R. D. D. Croy, jointly published by BIOS Scientific Publications Ltd (UK) and Blackwell Scientific Publications, UK.

Throughout the description and Examples, reference is made to the following sequences represented in the sequence listing:

SEQ ID NO. 1: nucleotide sequence of the 4-CL cDNA from Populus tomentosa;
SEQ ID NO. 2: amino acid sequence of the 4-CL protein from Populus tomentosa;
SEQ ID NO. 3: promoter region of the cinnamate-4-hydroxylase gene from Populus tomentosa;
SEQ ID NO. 4: forward primer for the PCR amplification of the 4-CL nucleotide sequence from Populus tomentosa;
SEQ ID NO. 5: reverse primer primer for the PCR amplification of the 4-CL nucleotide sequence from Populus tomentosa;
SEQ ID NO. 6: forward primer for detection of the chimeric C4H-4CL gene;
SEQ ID NO. 7: reverse primer for detection of the chimeric gene C4H-4CL.

### EXAMPLES

### Example 1

### Gene Cloning and Sequencing

Upon searching for the published poplar 4-CL gene sequences in NCBI database, Oligo6.0 software (commercially available) was used to design primers at both sides of gene encoding sequence (forward primer: [5′-ATGAATCCACAAGAAGAATTCATC-3′] SEQ ID NO. 4; and reverse primer: [5′-TTATATGCCTGCCAACTTTTCTTTCAG-3′] SEQ ID NO. 5), which were then used to amplify the corresponding sequence of 4-CL from Populus tomentosa.

1. mRNA extraction from Populus tomentosa.
RNA extraction (extract RNA by TRIZOL™ Kit)
100 mg plant material was ground in liquid nitrogen.
A. Add 1 ml TRIZOL, sit for 5 min at room temperature (20-25° C.).
B. Add 200 μl chloroform, agitate vigorously for 30 seconds, allow to remain at room temperature for 2 min.
C. Centrifuge at 12,000 g, 15 min, 4° C. Remove supernatant to a new tube, add 500 μl isopropanol, mix and allow to remain at room temperature for 15 min.
D. Centrifuge at 12,000 g, 15 min, 4° C. Discard supernatant, add 1 ml 70% alcohol.
E. Centrifuge at 7,500 g, 7 min, 4° C. Discard supernatant, air dry.
F. Resuspend in DEPC—H₂O.
   2. Use RevertAid H Minus First Strand cDNA Synthesis Kit (Fermentas) for reverse transcription of First Strand cDNA, following the manufacturer's instructions for operation.
   3. Perform PCR amplification using cDNA as template, the obtained gene sequence is as shown in SEQ ID NO. 1.

### Example 2

### Vector Construction

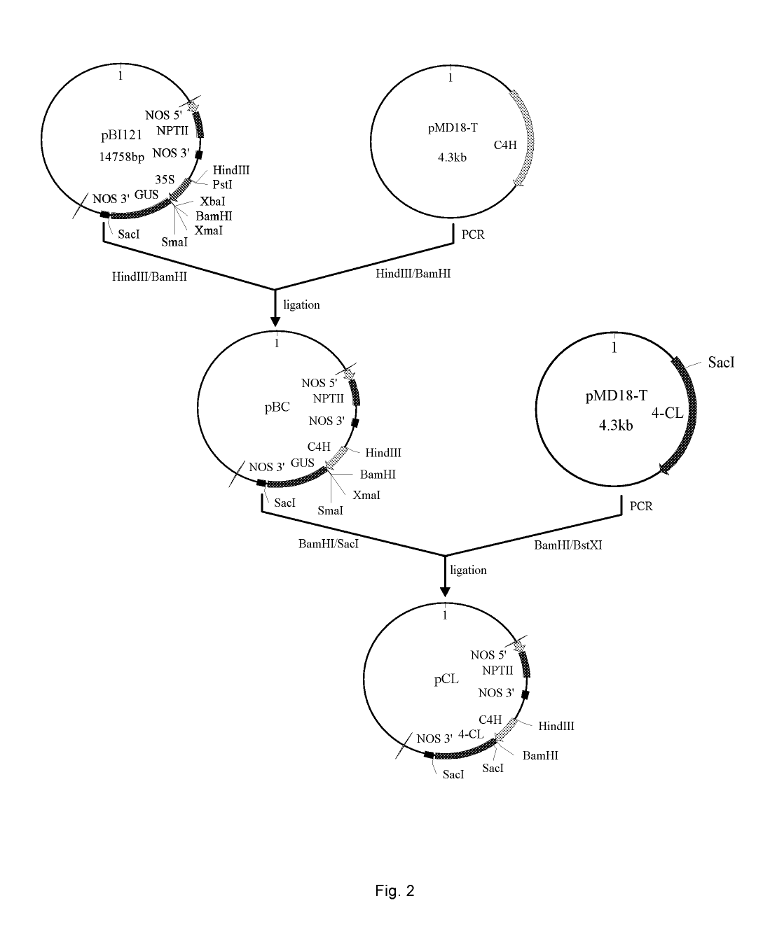

HindIII and BamHI restriction site sequences were introduced to 5′ end and 3′ end of C4H promoter (SEQ ID NO. 3) by PCR reaction. Then, the PCR amplification product of the C4H promoter and PBI121 plasmid were respectively digested by both restriction enzymes HindIII/BamHI. The digested fragment of PCR product and the digested longer fragment of PBI121 plasmid were ligated, and the resulting reaction product was used to transform competent cells E. coli DH5α (commercially obtained), and amplified. Then 4-CL gene was introduced into vector PBI121 to replace the original GUS sequence in PBI121, while the CaMV 35S promoter sequence formerly in the vector was replaced by C4H promoter sequence (as shown in FIG. 2). The inserted fragment was assessed by DNA sequencing.

### Example 3

### Transformation and Selection of the Plant

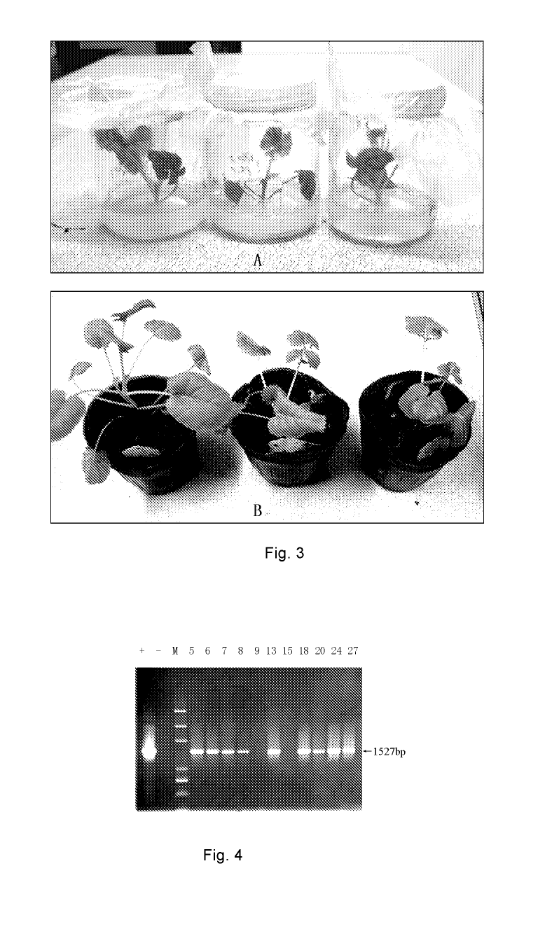

Agrobacterium LBA4404 transformation of Brassica napus
A. Preparation of aseptic seedlings: seed was soaked in 70% ethanol for 1 min, then in mercuric chloride (HgCl₂) for 13-15 min, rinsed 5 times with ddH₂O, plated in MS culture medium (PH 5.8), agar concentration of 0.8%. Aseptic Brassica napus seedling thus prepared was ready for use.
B. Transformation of Brassica napus cotyledon petiole: Agrobacterium tumefaciens LBA4404 (containing a T-DNA vector comprising the 4-CL gene operably linked to the C4H promoter as described in Example 2) was inoculated on solid LB medium. After 2 days, a single colony was picked into 50 ml YEP liquid medium (tryptone 10 g+yeast extract 10 g+NaCl 5 g+MgSO₄ 0.5 g) for culture. Cotyledon petioles were taken from aseptic seedling of 4-5 days, soaked in bacteria suspension for 5-8 min, with gently shaking in between. After that, the bacterial suspension was discarded and residual bacterial suspension on explant was removed with sterile filter paper. The resulting explant was placed on co-cultivation medium (MS+0.2 mg/L 6-benzyladenine (6-BA)+1 mg/L 2,4-Dichlorophenoxyacetic acid (2,4-D)+200 μm Acetosyringone (AS), PH5.8), and cultured for 2-3 days.
C. The co-cultivated explant was transferred to a different medium with carbenicillin (Car) only and without Kanamycin. Bacteria elimination and differential culture took place in dark greenhouse for 5-7 days. Then the co-cultivated explants were subjected to selection in selective medium with Kan selection pressure (MS+3 mg/L 6-BA+0.1 mg/L α-naphthaleneacetic acid (NAA)+5 mg/L silver nitrate (AgNO₃)+400 mg/L Car+15 mg/L Kan; pH 5.8), and wait for regenerated green buds to differentiate.
D. Regenerated buds were allowed to grow to a size of about 1 cm, cut off and transferred to root media (MS+0.2 mg/L NAA+10 mg/L Kan+400 mg/L Car; pH 5.8), followed by further selection on solid MS culture medium supplemented with Car and Kan (see FIG. 3A).
E. The rooted shoots were transferred outdoor, and then to soil containers after 10 days (see FIG. 3B).

### Example 4

### Nucleic Acid Analysis—PCR Assessment of the Transformed Plant

(1) Total DNA extraction was extracted from the transformed plant for PCR analysis according to the following protocol:
A. wipe the leaves with 70% alcohol, weigh out around 100 mg
B. add 600 μl extraction buffer (0.2 M Tris-Cl, 0.25 M NaCl, 25 mM EDTA, 0.5% SDS, pH 7.5), grind quickly at room temperature.
C. mix by vortexing for 5-10 s in 1.5 ml Ependorff tube.
D. Centrifuge at 12,000 rpm, 25 min, room temperature. Take supernatant, add isopropanol of same volume, precipitate overnight at −20° C.
E. Centrifuge at 12,000 rpm, 15 min, room temperature. Add 200 μl 70% alcohol, wash DNA precipitate.
F. Centrifuge at 12,000 rpm, 15 min, room temperature. Remove alcohol. Place tube upside down on paper towel, and wait for alcohol to evaporate.
G. Add 100 μl sterile water to resuspend DNA pellet. The concentration is determined by spectrophotometer or estimated by electrophoresis.
H. Perform PCR using total DNA as template.
   (2) PCR program

PCR reaction mixture was formulated the same as those for the PCR assessment of the plasmid (containing 4-CL gene). PCR primers were designed and synthesized based on the target gene and its upstream C4H promoter sequence of the plant expression vector: forward primer: [5′-CAGCTTCTTCTGCTTTCAATTACTCTC-3′] (SEQ ID NO. 6) and reverse primer [5′-CAACAGGAACTTCTCCCGCATT-3′] (SEQ ID NO. 7), time and temperature of the reaction were as follows:
94° C. 3 min,
94° C. 45 s,
59° C. 45 s,
72° C. 2 min 30 s, 30 cycles,
72° C. 5 min.

The test result shows that in positive control experiment and in samples from most transformed plant a DNA fragment of expected size could be amplified as shown by electrophoresis (pBI-C4H: 4CL: 1527 bp). In negative control the DNA fragment could not be amplified, which means that the genome of transgenic Brassica napus contained DNA fragment of said exogenous chimeric gene. The result is shown in FIG. 4.

### Example 5

### Assessment of Lignin Content, Sclerotinia sclerotiorum Resistance and Stem Strength of T0 Transgenic Line

### (1) Measurement of Lignin Content

Lignin content was measured according to the following protocol: A Stem part was cut off, and dried in oven till it reached constant weight, pulverized thoroughly by pulverizer; then 0.3 g of the sample was weighed out, to which 7.5 ml 72% sulphuric acid was added, and digested for 1 h at 30° C. The digestion solution was diluted with distilled water till the concentration of sulphuric acid is 4%, following treatment for 1 h at 121° C., the sample was cooled to room temperature, filtered with one layer of filter paper; the residue was dried and weighed, and lignin content was calculated. Among 31 (PCR positive) 37301 T0 transgenic lines obtained, some of the transgenic lines were randomly selected for lignin content measurement (see table 1) in this experiment. As can be seen from the table, the lignin contents of transgenic lines 6, 13, and 18 were all increased compared to control, whereby lines 6 and 13 had the highest increase magnitude, which increase amount to 13.41% and 11.48% based on the lignin content of the control, respectively.

TABLE 1
lignin content analysis of transgenic Brassica napus

| | CK | 6 | 13 | 18 |
|---|---|---|---|---|
| Lignin content | 9.32 ± 0.69 | 10.57 ± 0.52 | 10.39 ± 0.72 | 10.35 ± 0.59 |
| (%) | | | | |

(2) Assessment of Sclerotinia sclerotiorum Resistance

The Sclerotinia resistance test was conducted by inoculating in vitro a Brassica napus leaf with Sclerotinia sclerotiorum at the seedling stage. Before bolting of the Brassica napus, leaves were excised from the same spot, put on white porcelain plates, with two sets on each plate, and 8 leaves each set, petioles were wrapped with wet gauze to stay moisturized. Sclerotinia mycelia pieces were invertedly deposited on mesophyll at upper part of the main vein of the leaves. After inoculation, leaves were covered with transparent plastic film to stay 100% moisturized. After incubation for 2 days at 23° C., the diameter (cm) of the lesions was measured. Measurement was repeated three times, each time repeated with 5-7 leaves, and average area of their lesions was calculated. The result shows that, compared with non-transgenic plant as control, transgenic lines 6, 13, 18 have lesions with decreased extension area (P [symbol] α=0.05 [symbol] , which are respectively 66.42%, 77.87%, 72.08% of the control (see FIG. 5).

### (3) Test for Stem Strength

During the maturation period of Brassica napus, the stem strength of Brassica napus was determined by lodging tester DIK-7401 (purchased from Japan). Normally growing Brassica napus was selected, with 15 plants in each region. The upper canopy part at the middle part of stem was cut off, the plant was bend to a certain angle (45°) by lodging tester to measure stem strength (N/stem), and calculate the average value. The result shows that, the stem strength increases compared to control (P [symbol] α=0.05 [symbol] , and the increase magnitude are 27.58%, 17.16%, 22.84%, respectively (see FIG. 6).

1. A method of increasing the resistance against fungal disease or of increasing the lodging resistance in a Brassica plant comprising introducing a chimeric gene into cells of said Brassica plant, said chimeric gene comprising a nucleotide sequence encoding a 4-coumarate-CoA ligase operably linked to a heterologous plant-expressible promoter, a transcription termination and a polyadenylation region.

2. The method of claim 1, wherein said plant-expressible promoter is a wound-inducible plant-expressible promoter or a xylem selective plant-expressible promoter.

3. (canceled)

4. The method of claim 1, wherein said plant-expressible promoter comprises the nucleotide sequence of SEQ ID No.: 3.

5. The method of claim 1, wherein said 4-coumarate-CoA ligase comprises the amino acid sequence of SEQ ID No.: 2.

6. The method of claim 1, wherein said nucleotide sequence encoding a 4-coumarate-CoA ligase comprises the nucleotide sequence of SEQ ID No.: 1 from the nucleotide at position 11 to the nucleotide at position 1621, or comprises the sequence of SEQ ID No.: 1.

7. The method of claim 1, wherein said fungal disease is caused by Sclerotinia sclerotiorum.

8. The method of claim 1, wherein said Brassica plant is Brassica napus, Brassica junceae, Brassica oleraceae, Brassica rapa, Brassica nigra, or Brassica carinata.

9. A chimeric DNA comprising a nucleotide sequence encoding a 4-coumarate-CoA ligase, operably linked to a heterologous plant-expressible promoter, a transcription termination, and a polyadenylation region.

10. The chimeric DNA of claim 9, wherein said promoter is a wound-inducible plant-expressible promoter or a xylem-selective plant-expressible promoter.

11. (canceled)

12. The chimeric DNA of claim 9, wherein said promoter comprises the nucleotide sequence of SEQ ID No.: 3.

13. The chimeric DNA of claim 9, wherein said 4-coumarate-CoA ligase comprises the amino acid sequence of according to SEQ ID No.: 2.

14. A Brassicaceae plant cell, plant, or seed comprising a chimeric DNA of claim 9.

15. (canceled)

16. (canceled)

17. An isolated DNA molecule comprising the nucleotide sequence of SEQ ID No.: 1.

18. A method of obtaining resistance in a Brassica plant against Sclerotinia sclerotiorum comprising introducing the chimeric gene of claim 9 or an isolated DNA molecule comprising nucleotide sequence of SEQ ID No.: 1 into cells of a Brassica plant.

19. A method of increasing lodging resistance of Brassica plants comprising introducing the chimeric gene of claim 9 or an isolated DNA molecule comprising nucleotide sequence of SEQ ID No.: 1 into said plants.

20. (canceled)

21. The method of claim 1, wherein 4-coumarate-CoA ligase is from a plant.

22. The chimeric DNA of claim 9, wherein 4-coumarate-CoA ligase is from a plant.

23. The Brassicaceae plant cell, plant or seed of claim 14, which is a Brassica plant cell, plant, or seed.

24. The method of claim 18, wherein the Brassica plant is Brassica napus.

25. The method of claim 19, wherein the Brassica plant is Brassica napus.


SEQUENCE LISTING (7 sequences)

SEQ ID NO: 1 - DNA, 1631 bp, Artificial Sequence
  cDNA nucleotide sequence of the 4-coumarateCoA ligase from Populus tomentosa
cgccggatccatgaatccacaagaagaattcatctttcgctcaaaattaccagacatctacatcccgaaaaaccttcccctgcattcatacgttcttgaaaacttgtctaaccattcatcaaaaccttgcctgataaatggggcgaatggagatgtctacacctatgctgacgttgagctcacagcaagaagagttgcttctggtcttaacaagattggtattcaacaaggtgacgtgatcatgctcttcctaccaagttcacctgaattcgtgcttgctttcctaggcgcttcacacagaggtgccattatcactgctgccaatcctttctccacccctgcagagctagcaaaacatgccaaggcctcgagagcaaagcttctgataacacaggcttgttactacgagaaggttaaagattttgcccgagaaagtgatgttaaggtcatgtgcgtggactctgccccggatgggtgcttgcacttttcagagctaacacaggcagacgaaaatgaagcgcctcaggtcgacattagtcccgatgatgtcgtagcattgccttattcatcagggactacagggttgccaaaaggggtcatgttaacgcacaaagggctaataaccagtgttgctcaacaggtagatggagacaatcctaacctgtattttcacagtgaagatgtgattctgtgtgtgctgcctatgttccatatctatgctctgaattcaataatgctctgcgggctgagagtcggtgcctcgattttgataatgccaaagtttgagattggttctttactgggattgattgagaagtacaaggtatctatagcaccggttgttccacctgtgatgatgtcaattgctaagtcacctgatcttgacaagcatgacttgtcttctttgaggatgataaaatctggaggggctccattgggcaaggaacttgaagatactgtcagagctaagtttcctcaggctagacttggtcagggatatggaatgaccgaggcaggacctgttctagcaatgtgcttggcatttgccaaggaaccattcgacataaaaccaggtgcatgtgggactgtagtcaggaatgcagagatgaagattgttgacccagaaacaggggcctctctaccgaggaaccagcctggtgagatctgcatccggggtgatcagatcatgaaaggatatcttaatgaccctgaggcaacctcaagaacaatagacaaagaaggatggctgcacacaggcgatatcggctacattgatgatgatgatgagcttttcatcgttgacagattgaaggaattgatcaagtataaagggtttcaggttgctcctgctgaactcgaagctctgttaatagcccatccagagatatccgatgctgctgtagtaggattgaaagatgagaatgcgggagaagttcctgttgcatttgtagtgaaatcagaaaagtctcaggccaccgaagatgaaattaagcagtatatttcaaaacaggtgatattctacaagagaataaaacgagttttcttcattgaagctattcccaaggcaccatctggcaagatcctgaggaagaatctgaaagaaaagttggcgggcatataagagctcctcg

SEQ ID NO: 2 - protein, 536 aa, Artificial Sequence
  Synthetic Construct
MNPQEEFIFRSKLPDIYIPKNLPLHSYVLENLSNHSSKPCLINGANGDVYTYADVELTARRVASGLNKIGIQQGDVIMLFLPSSPEFVLAFLGASHRGAIITAANPFSTPAELAKHAKASRAKLLITQACYYEKVKDFARESDVKVMCVDSAPDGCLHFSELTQADENEAPQVDISPDDVVALPYSSGTTGLPKGVMLTHKGLITSVAQQVDGDNPNLYFHSEDVILCVLPMFHIYALNSIMLCGLRVGASILIMPKFEIGSLLGLIEKYKVSIAPVVPPVMMSIAKSPDLDKHDLSSLRMIKSGGAPLGKELEDTVRAKFPQARLGQGYGMTEAGPVLAMCLAFAKEPFDIKPGACGTVVRNAEMKIVDPETGASLPRNQPGEICIRGDQIMKGYLNDPEATSRTIDKEGWLHTGDIGYIDDDDELFIVDRLKELIKYKGFQVAPAELEALLIAHPEISDAAVVGLKDENAGEVPVAFVVKSEKSQATEDEIKQYISKQVIFYKRIKRVFFIEAIPKAPSGKILRKNLKEKLAGI

SEQ ID NO: 3 - DNA, 1117 bp, Artificial Sequence
  Promoter region of the cinnamate-4-hydroxylase gene from Populus tomentosa
ctctgcagccaagttagccagacatgaaaaggtgagaaaaacattttttgacataagaaactaacgttgcttggatttaagatagaattattgaccttgtaagaagacaaacgtattcttggaaattaacatgtattaataaatgcaaagtagtttgtcacactatgggataaaatctctaataaaaagtaagaccttatagtttcaagaggttaggttgatatttaaagagagatttcttttataactttttaggttgaaatcttgaaattaatattaaaaagatttgttaatcattttcttttaaatactttggattgatgccaggggtttacatttaaaattattcttaaatgaaacttgatggtgtgtgtttgatattgtattttcacatgtttttaaaatgaatttgtttttaaaaaatatcaaattaataatttttttattttttttcaaagattttaatgtattaattttaaaaataaaataaaaattattttaatatatttttaaataaaaaatattttcaaataaaaaaattaaaaaataaaaagataggaaaaaaactttcttggttgagggcctcattccttgaaatttaagaaagcgtttgggaactcggtgcaaaccgtgttcctattaaatttgaatttttttttgttaaaatttaatgcggtttatattttttggatcgttttgatgtgctgatatcaaaaataattttaaaaaaataaaaaaaattattaatatgtatttcaacacgaaaagttatttgaaaaacaatcaccaccccattgattcacacgttctaaattatcataaattagtggcgcccacattacatcttgaatagaaatacaaaaaggccaggcaaattaattaatatggtgaccatatgacattttcagccaccaacccgccttacctactactatccatgattatcaataacagtctcctaccacctcaaatgtaacgccgttaactctctctctctcccccacacacacaacccaacgcgtgaaattcaacttcatttcctctctaatttttgcagcttataaaacccaagctctcctcatcctgttgctcccatccaaccatcactctctagaac

SEQ ID NO: 4 - DNA, 24 bp, Artificial Sequence
  Forward primer for the PCR amplication of the 4CL gene of Populus tomentosa
atgaatccacaagaagaattcatc

SEQ ID NO: 5 - DNA, 27 bp, Artificial Sequence
  Reverse primer for the PCR amplication of the 4CL gene of Populus tomentosa
ttatatgcctgccaacttttctttcag

SEQ ID NO: 6 - DNA, 27 bp, Artificial Sequence
  Forward primer for detection of the chimeric C4H-4CL gene
cagcttcttctgctttcaattactctc

SEQ ID NO: 7 - DNA, 22 bp, Artificial Sequence
  Reverse primer for detection of the chimeric gene C4H-4CL
caacaggaacttctcccgcatt